(19) United States
(12) Patent Application Publication
(10) Pub. No.: US 2019/0310819 A1
(43) Pub. Date: Oct. 10, 2019

(54) AUGMENTED REALITY IMAGE DISPLAY SYSTEMS AND METHODS

(71) Applicant: Carto Technologies, LLC, St. Louis, MO (US)

(72) Inventors: Jimmy Xu, St. Louis, MO (US); Austin Sloop, St. Louis, MO (US); Peng Hu, St. Louis, MO (US); Tara Gildersleeve, Carlsbad, CA (US)

(21) Appl. No.: 16/379,968

(22) Filed: Apr. 10, 2019

Related U.S. Application Data
(60) Provisional application No. 62/655,274, filed on Apr. 10, 2018.

(51) Int. Cl. G06F 3/14; G06T 7/70; H04N 5/272; G16H 30/40

(52) U.S. Cl. CPC G06F 3/1454; G06T 7/70; H04N 5/272; G06F 3/017; G06T 2207/10016; G06T 2207/30196; G16H 30/40

(57) ABSTRACT

This invention is an augmented reality device that displays virtual screens at user specified positions taking inputs from the electronic medical record, PACs system, and imaging device. The virtual screens can be displayed in any orientation. Machine learning tools will improve ease of workflow. Collaboration tools will be available. An API will ease interoperability between radiology imaging devices and augmented reality systems. Devices such as catheters/needles impregnated with radiopaque markers and other radiopaque target markers are also discussed.

### CROSS-REFERENCE TO RELATED APPLICATIONS

This application claims priority to U.S. Provisional Patent Application Ser. No. 62/655,274, filed on Apr. 10, 2018, entitled “AUGMENTED REALITY IMAGE DISPLAY SYSTEM,” currently pending, the entire disclosure of which is incorporated herein by reference.

### BACKGROUND

Minimally invasive surgical techniques such as laparoscopy, endovascular and interventional radiology have obvious benefits in minimizing tissue damage. The main problems exist in disruption of the feedback loops to the operator. Visual feedback is obstructed by the patient's tissue and so the operator is unable to directly view the surgical site.

Current methods for visualizing the surgical site include cameras or noninvasive imaging modalities, most commonly ultrasound and fluoroscopy. These devices output visual information to a screen that is either physically attached to the imaging device (e.g., ultrasound), or is displayed on a screen attached to a boom. While the resolution of these screens can be satisfactory there are numerous limitations to these screens.

First, the screens are not in the same orientation as the patient. In endovascular procedures the patient lies horizontally on the procedure table while the screen is upright. Thus the operator views an upright screen while intervening on a horizontal patient. This causes increased mental effort to geometrically orient the image onto the patient as well as context switching as the operator must constantly reorient themselves toward the patient which in it of itself increases task time and decreases accuracy. Moreover, the large distance between the screen and the patient also leads to eye accommodation and conversion leading to visual fatigue. The combination of these factors adversely impacts task performance.

A second issue is ergonomics. Work related musculoskeletal disorders receive little attention, but can cause significant morbidity. When screens are attached to the machines themselves they are constrained by limited space in the operating room. This can lead to poor placement of the screens. When screens are attached to a boom often they cannot be lowered sufficiently. In addition, with multiple people in the room not everyone's preferences can be accommodated. Musculoskeletal conditions for operators can lead to significant morbidity and economic cost from lost workdays. These can in turn impact patient welfare.

Surgical operating theaters are sterile environments to limit the risk of infection and harm to the patient. The process of interacting with the imaging information however is not sterile. Thus, operators are unable to access crucial patient and imaging information. They must either ask another person in the room to display the information or they must un-glove, break the sterile field, and then re-glove. This process is time consuming, costly, and can be ultimately harmful to the patient. Another unintended consequence of the mandate to maintain sterility is that collaboration between surgeons and surgical staff is often limited. Since there is no way to interact with the imaging devices/information without breaking sterility, operators using conventional techniques require workarounds that cause distraction and context switching, but more seriously can risk inadvertent voiding of sterility which can cause increased infection.

Categories of conventional systems include projector-based, boom- and/or gantry-based, and headset displays. Conventional projector-based systems use a mounted video projector that displays images onto the patient. Conventional boom- and/or gantry-based devices use a monitor that is positioned over the patient that can be used for surgical planning or intraoperative guidance. Conventional headset-based devices use a video see-through display. However, these conventional systems are mostly static and still require patient information and surgical data to be displayed on other screens. A barrier to widespread adoption of the above techniques is that their operation is not intuitively integrated into the end user's workflow. Image registration and manipulation must be accomplished pre-operatively or by a non-sterile technologist intraoperatively. Using projectors or screens mounted to gantries are cumbersome enough to prevent regular use.

### SUMMARY

An aspect of the invention includes an augmented reality (AR) system that creates interactive virtual screens for surgical collaboration. The system interfaces with the image output from the imaging device and displays the information on a virtual screen. The system is operable to receive multiple inputs from different imaging devices and integrate them into an augmented workspace for the operator. The system is operable to be manipulated (e.g., by an operator) via techniques that do not require touch. In exemplary embodiments, the system is manipulated using one or more of eye, gestures, speech, and other methods of control. In further exemplary embodiments, the system enables collaboration (e.g., when multiple operators are in the same room) via annotations. In yet further exemplary embodiments, the system includes machine-learning models and/or other artificial intelligence techniques to guide the operator, such as medical registration and segmentation, and image processing, for example.

### DESCRIPTION OF THE SEVERAL VIEWS OF THE DRAWINGS AND APPENDIX

In the accompanying drawings, which form a part of the specification and are to be read in conjunction therewith in which like reference numerals are used to indicate like or similar parts in the various views:

FIG. 1 illustrates an exemplary system architecture.

FIG. 2 illustrates an exemplary virtual screen setup.

FIG. 3 illustrates an exemplary user interface design. The main view screen is delineated by the rectangular box surrounding the figures.

FIG. 4 illustrates machine learning incorporated into the image display.

FIG. 5 illustrates an exemplary method of selecting a virtual screen.

FIG. 6 illustrates an exemplary method of rotating/scaling.

FIG. 7 illustrates an exemplary method of rotating along the longitudinal axis and translation.

FIG. 8 illustrates an exemplary method of inactivating the virtual screens.

FIG. 9 illustrates an exemplary collaboration between operators and staff.

FIG. 10 illustrates an exemplary implementation of machine learning system.

FIG. 11 illustrates an exemplary application-programming interface.

FIG. 12 illustrates a 3D printed catheter design with radiopaque image targets.

FIG. 13 illustrates an alternative design using radiopaque markers.

FIG. 14 illustrates another alternative design using radiopaque markers.

FIG. 15 illustrates the 3D printed catheter or needle design with radiopaque image targets in use.

FIG. 16 illustrates the machine-learning algorithm identifying the radiopaque image targets on the x-ray image.

FIG. 17 illustrates the augmented reality system overlaying the image onto a radiopaque marker placed on top of the patient within the sterile field.

FIG. 18 illustrates an example of a marker rotated in three-dimensional space. Two angles of note are indicated. ω represents a rotation about the central line of the device, in reference to an arbitrary standard. φ represents a rotation of the central axis from some other plane, which can be arbitrarily chosen. The bottom portion labeled “Areas vs Angles” illustrates the measurable area of each sub marker for the example device in FIG. 18, with darker regions indicating more visible area and lighter regions indicating less visible area of each subsection. The width of each section corresponds to a rotation orthogonal from the central axis, φ, and the vertical axis is representative of 360* of rotation around the central longitudinal axis, ω.

FIG. 19 illustrates the compression of the example marker in FIG. 18 compressed to two dimensions as if taken by a medical imaging device.

FIG. 20 is a block diagram of an exemplary embodiment of a computer system upon which embodiments of the inventive subject matter can execute.

Appendix A provides one exemplary embodiment of details regarding image processing.

### DETAILED DESCRIPTION

In the following detailed description of example embodiments, reference is made to the accompanying drawings that form a part hereof, and in which is shown by way of illustration specific example embodiments in which the inventive subject matter may be practiced. These embodiments are described in sufficient detail to enable those skilled in the art to practice the inventive subject matter, and it is to be understood that other embodiments may be utilized and that logical, mechanical, electrical and other changes may be made without departing from the scope of the inventive subject matter.

Some portions of the detailed descriptions which follow are presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of steps leading to a desired result. The steps are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the following discussions, terms such as “processing” or “computing” or “calculating” or “determining” or “displaying” or the like, refer to the action and processes of a computer system, or similar computing device, that manipulates and transforms data represented as physical (e.g., electronic) quantities within the computer system's registers and memories into other data similarly represented as physical quantities within the computer system memories or registers or other such information storage, transmission or display devices.

The description of the various embodiments is to be construed as describing examples only and does not describe every possible instance of the inventive subject matter. Numerous alternatives could be implemented, using combinations of current or future technologies, which would still fall within the scope of the claims. The following detailed description is, therefore, not to be taken in a limiting sense, and the scope of the inventive subject matter is defined only by the appended claims.

In some aspects, the systems, methods, and techniques described herein provide virtual interactive and dynamic screens that display vital information and can be placed at any user selected position. In an exemplary embodiment, the systems include a touchless user interface that may include eye tracking, gesture control, and/or voice control (e.g., to maintain sterility). The systems, methods, and techniques described herein include a control system that does not interfere with the operator's ability to perform the procedure or surgery. In a further exemplary embodiment, the systems include one or more interactive virtual screens that can be shared among users/operators in a procedural suite or operating room because teams perform these procedures. In yet further exemplary embodiments, the systems, methods, and techniques described herein include collaboration tools (e.g., for preoperative and/or intraoperative planning, etc.).

Conventional systems are specific to a certain procedure and cannot be generalized. For specialties such as interventional radiology, however, operators perform procedures in multiple anatomical locations and it is necessary to have an AR system that can be adapted to use in other areas. In some exemplary embodiments, the systems, methods, and techniques described herein are adaptable to usage in a plurality of procedures, locations, areas, and the like.

Additionally, specialties such as interventional radiology often use multiple imaging modalities from multiple vendors to accomplish a specific procedure. In some exemplary embodiments, the AR systems, methods, and techniques described herein are configured to accommodate multiple inputs simultaneously and allow users to manipulate them in a seamless way.

In further embodiments, the systems, methods, and techniques described herein enable interoperability for medical apps. In an exemplary embodiment, the systems, methods, and techniques described herein include an application-programming interface (API) that enables an application for a specific headset or medical device to be ported to another type of headset or medical device.

In yet further embodiments, the systems, methods, and techniques described herein are operable to analyze information recorded during a procedure (e.g., images, radiation exposure data, power output data, etc.) in clinically and/or operationally useful ways.

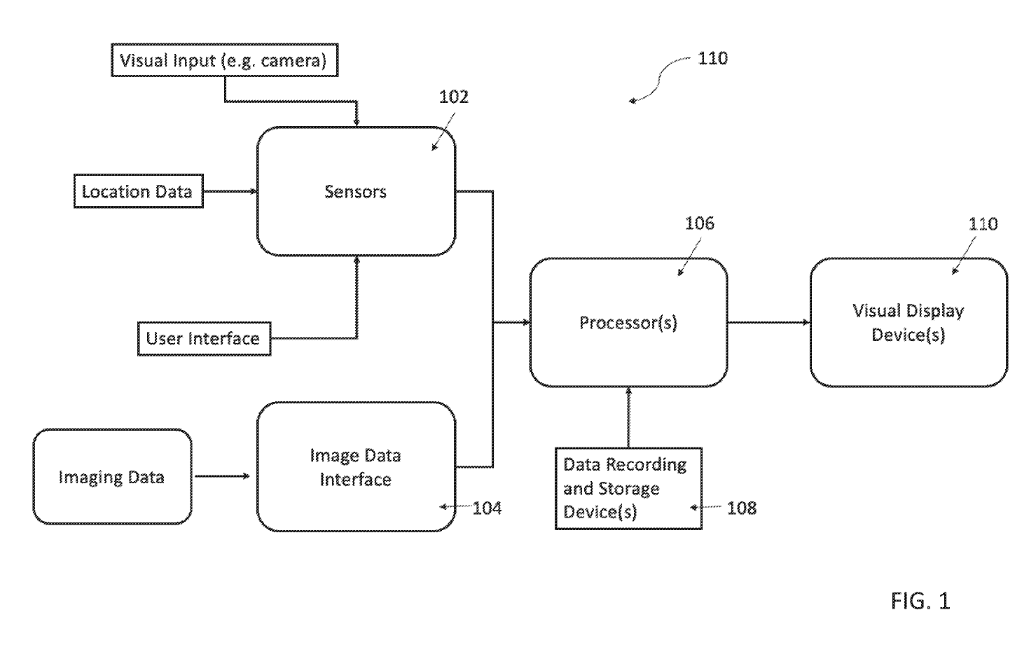

FIG. 1 illustrates an exemplary embodiment of a system 100 comprised of a plurality of sensors 102, an image data interface 104, one or more processors 106, data recording and storage device(s) 108, and visual display device(s) 110. The sensors 102 and the image data interface 104 are each electrically and/or communicatively coupled to the processor(s) 106. The processor(s) 106 are electrically and/or communicatively coupled to the data recording and storage device(s) 108 and the visual display device(s) 110. The electrical and/or communicative couplings of the components of system 100 may include devices and techniques capable of facilitating the exchange of data. The electrical and/or communicative couplings may include, at least in part, communications networks that facilitate the exchange of data, such as those that operate according to the IEEE 802.3 (e.g., Ethernet), IEEE 802.11 (e.g., Wi-Fi), and/or IEEE 802.15 (e.g., Bluetooth, ZigBee, etc.) protocols, for example. In another embodiment, the electrical and/or communicative couplings may include any medium that allows data to be physically transferred through serial or parallel communication channels (e.g., copper wire, optical fiber, computer bus, wireless communication channel, etc.).

Exemplary sensors 102 include sensors that are operable to input visual feedback (e.g., a camera, etc.), haptic feedback (e.g., a touchscreen device, etc.), as well as imaging data into the processor(s) 106 (e.g., centralized processing server, etc.). A sensor is a broad term that is intended to encompass its plain and ordinary meaning including without limitation cameras, infrared cameras, stereoscopic cameras, heartbeat sensor, touch sensor, humidity sensor, gas sensor, smoke sensor, thermistor, ultrasonic sensor, etc.

The image data interface 104 is operable to receive imaging data from one or more imaging devices (e.g., X-ray devices, magnetic resonance imaging (MM) devices, etc.) and/or computer-readable media devices on which imaging data is stored and communicate the imaging data to the processor(s) 106. Imaging data is also intended to be used in its plain and ordinary meaning to include raw source data directly from the machine or as video output obtained through a frame grabber or streamed via camera uplink.

In some embodiments, the processor(s) 106 comprise the visual display devices 110. In other embodiments, the visual display device(s) 110 are tethered to the processor(s) 106 in a wired and/or wireless fashion. The central processing unit may have multiple graphical processing unit cores or any other configuration. Visual display is a broad term that is intended to encompass its plain and ordinary meaning. In some embodiments the visual display may be a head mounted device (camera pass through device, optical see through device), or a display system in some other form.

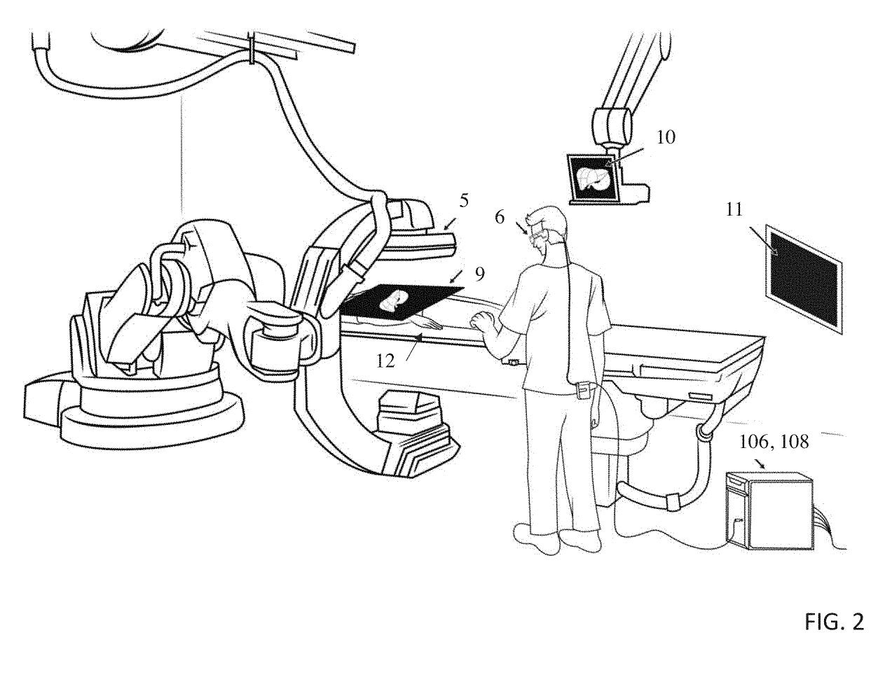

FIG. 2 demonstrates an exemplary embodiment of system 100 implemented in an operating room environment. In this exemplary setup, a patient 12 lies on a table in front of an operator 34 but may be in any other configuration including, but not limited to, prone, seated, lateral decubitus, etc. In some embodiments, the operator 34 will don a headset 6 comprising the visual display device 110 that is electrically and/or communicatively coupled (e.g., tethered) to a computing device comprising processor(s) 106 and/or data recording and storage device(s) 108. In still other embodiments, the headset 6 may communicate wirelessly with the processor(s) 106 or may incorporate the processor(s) 106. In other embodiments, a headset may not be necessary if the holograms or augmented visual objects can be implemented without one. For example in these embodiments, the visual display device 100 may comprise an electronic contact lens or the like. The processor(s) 106 receive inputs from an imaging machine 5 (e.g., via the image data interface 104) and other sources including but not limited to the electronic medical record, other imaging modalities, and the like (e.g., via sensors 102, image data interface 104, and/or data recording and storage device(s) 108, etc.).

In an embodiment, image registration occurs using an image target (also referred to as a target marker in some embodiments) that functions as a fiducial marker for emplacement of the image or video. Emplacement may refer to pose, position, orientation or any other appropriate locational information. There can be a single image target or multiple image targets. In some embodiments, this image target may include but is not limited to a radioopaque sticker, lenticular array, or other object. In other embodiments, the image target may include anatomical markers which include but are not limited to the nipples, umbilicus, spine bones, ribs, shoulder, hips, pelvis, femur, or any other appropriate anatomical marker. Additionally, an image target is not necessary for emplacement, and emplacement may occur using other forms. Emplacement may also arise from any other number of tracking methods including but not limited to electromagnetic field. Other embodiments may include and are not limited to video feed streamed to a web server (e.g., through a WebRTC protocol, etc.) that is then processed on the device used by the operator 34 (e.g., processor(s) 106 and/or headset 6, etc.) to display the images. The video feed may be streamed to a local server computing device or directly fed into the device used by the operator 34. The inputs 8 may be wireless or physical wired connections. The tether connecting headset 6 and processor(s) 106 may be wired or wireless.

In this embodiment the virtual screen 9 live streams the image output from the imaging machine 5. In an embodiment, a virtual screen is an augmented reality object that displays information. The streaming may be via methods including, but not limited to, using a real time streaming protocol (RTSP) over a WebRTC, HTTP Live streaming, or any other image and/or video streaming protocol. Additionally, the stream may be hardwired to the processor(s) 106 and then streamed to the virtual object. Virtual screens 10 and 11 are exemplary embodiments of other uses for screens that include but are not limited to the electronic medical record and/or prior imaging. The differences between virtual screens 9, 10, and 11, are the type of content within the screens as well as the orientation angle.

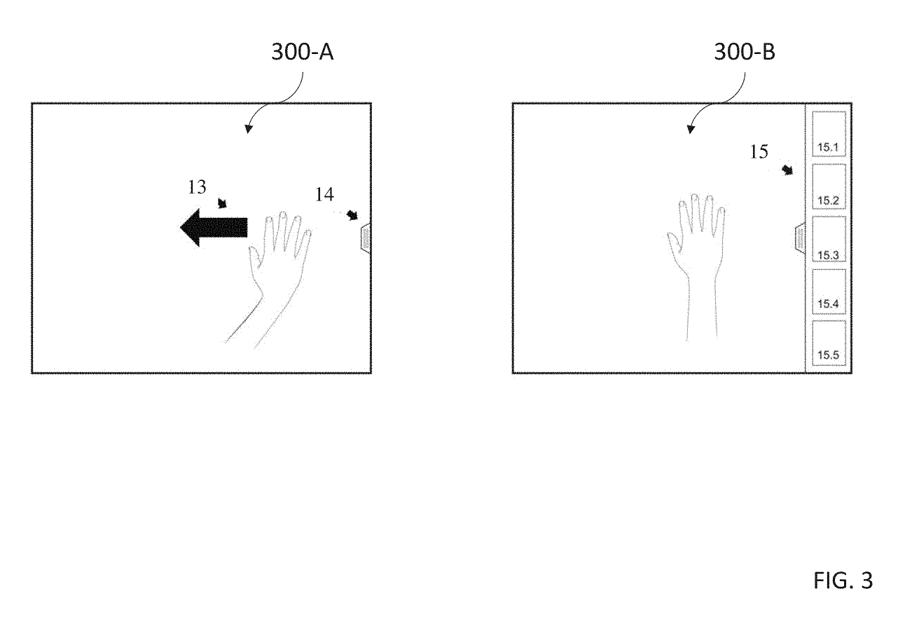

FIG. 3 illustrates a main view screen in which virtual screens containing images, videos, livestream, or other forms of media and/or including guidance are emplaced. The view screen 300-B includes a repository 15 of all the media items (e.g., in a thumbnail format, icons, etc.) that have been created during the procedure. The view screen 300-B enables the user (e.g., operator, surgeon, etc.) to scroll through the media items and view one or more of the items on the view screen as well as emplace it in any location using gesture control 13, as shown in view screen 300-A. In other embodiments, the repository 15 including the media items may be hidden with a tab 14 and only show up with a certain gesture, emplaced in other screen positions or other orientations. Voice control may be used and voice recordings and other annotations may be added during the procedure.

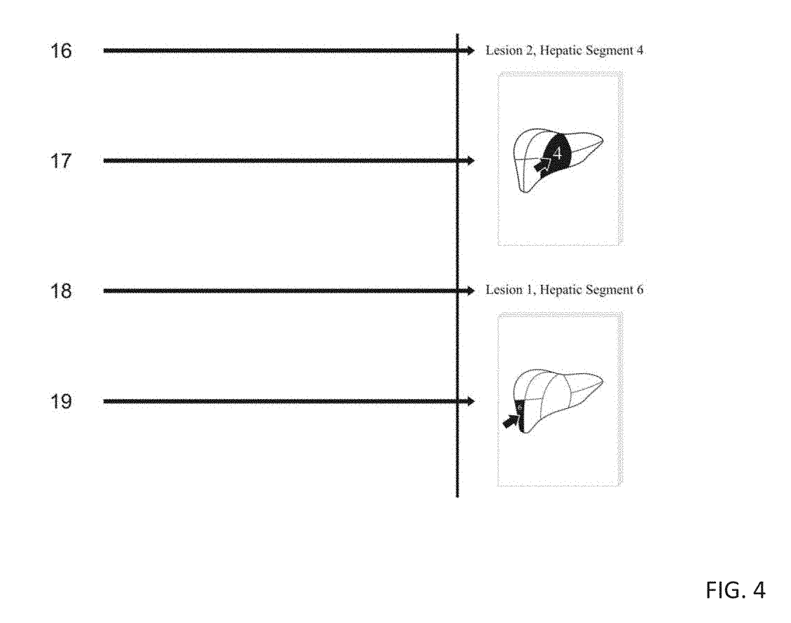

FIG. 4 illustrates a use case for machine learning tools incorporated into the organization of the media created in the surgery. Machine learning tools will be able to recognize the stage of the surgery and help categorize 16, 18 the media for fast retrieval. Additionally the media can be ordered chronologically 17, 19 or in any other order that the user desires. In other embodiments, the machine learning tools will also be able to recognize the angle of the images and display it at the angle it was captured, identify anatomical landmarks, calculate ratios and other procedural guidance metrics yet to be named.

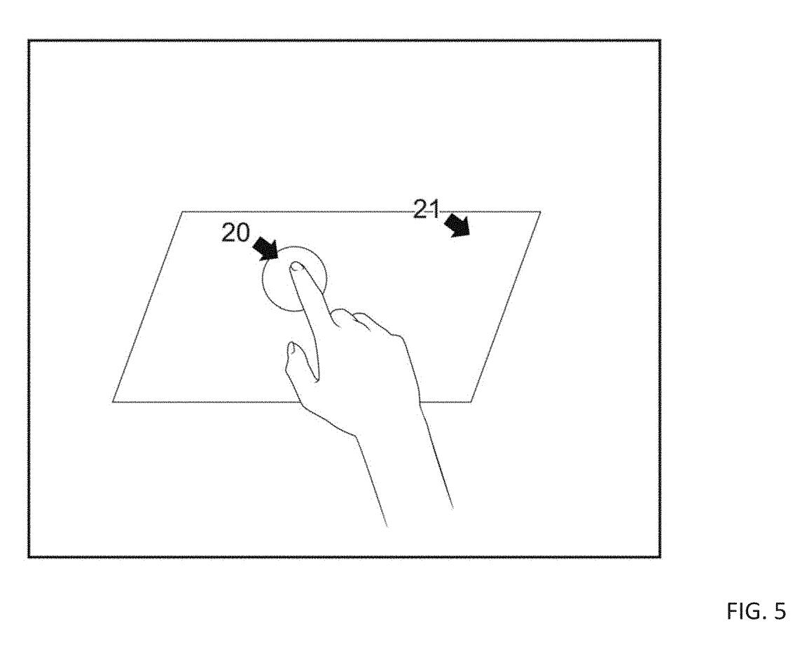

FIG. 5 illustrates an exemplary method of selecting the virtual screen to isolate a region of data in the field of view of the optical sensors and/or allow a user to designate a set of information being provided and save or modify such data. In some embodiments, this is performed by pointing at the virtual screen 21 with a finger 20 with visual feedback (e.g., via visual display device(s) 110) to indicate when the item is selected and able to be manipulated. A single image or stack of a plurality of images may be emplaced onto a virtual screen.

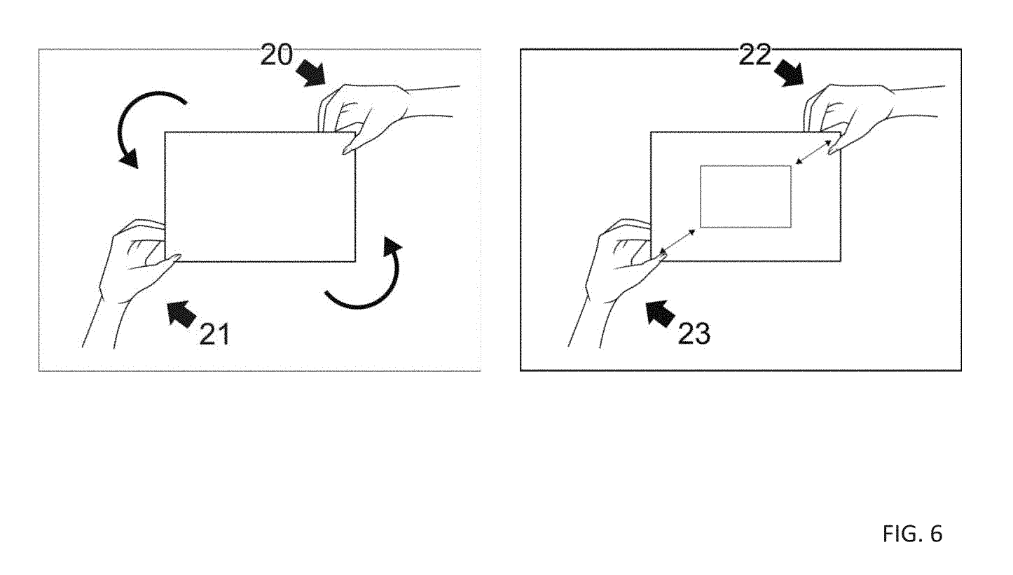

FIG. 6 is an exemplary figure demonstrating the ability to manipulate the image provided to the operator to manually change the orientation or visualization of data provided. In some embodiments rotating the image can be accomplished by selecting a corner and/or diagonal corners 20, 21. In other embodiments, the pinch and zoom method 22, 23, using thumb and index finger, can be used. In this embodiment a set or subset of data may be selected and adjusted to fill the available display, thus showing more detail and information.

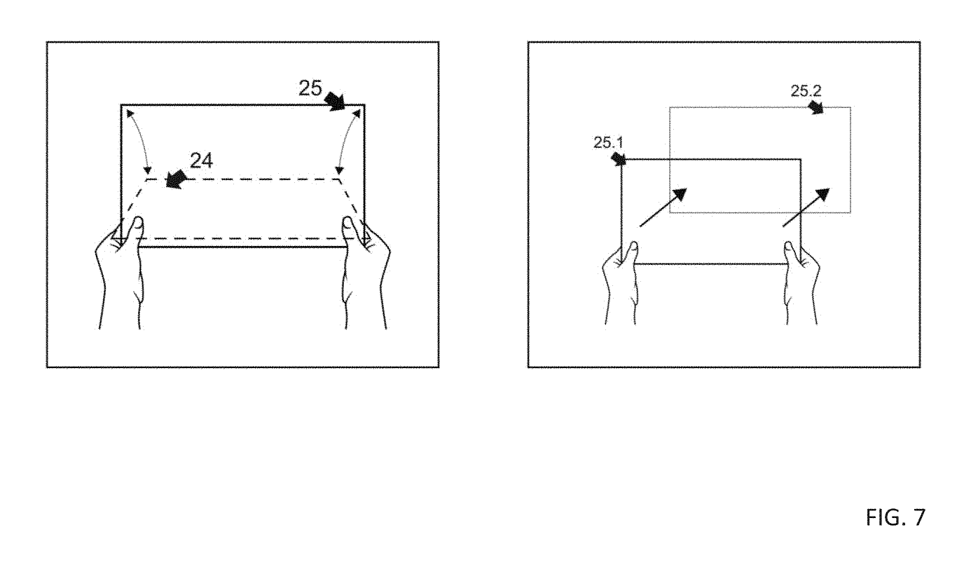

In FIG. 7, an exemplary figure showing the rotation of the virtual screen along the longitudinal axis. 24, 25. This will allow the operator to precisely define the best imaging plane. Translation of the image can also be accomplished using natural gestures which include grabbing both sides of the screen and angling it forward, or pushing the whole screen 25.1, 25.2. In other embodiments this and other forms of manipulation as outlined above may be accomplished using other methods including, but not limited to virtual buttons, and other gestures, voice control, and eye control.

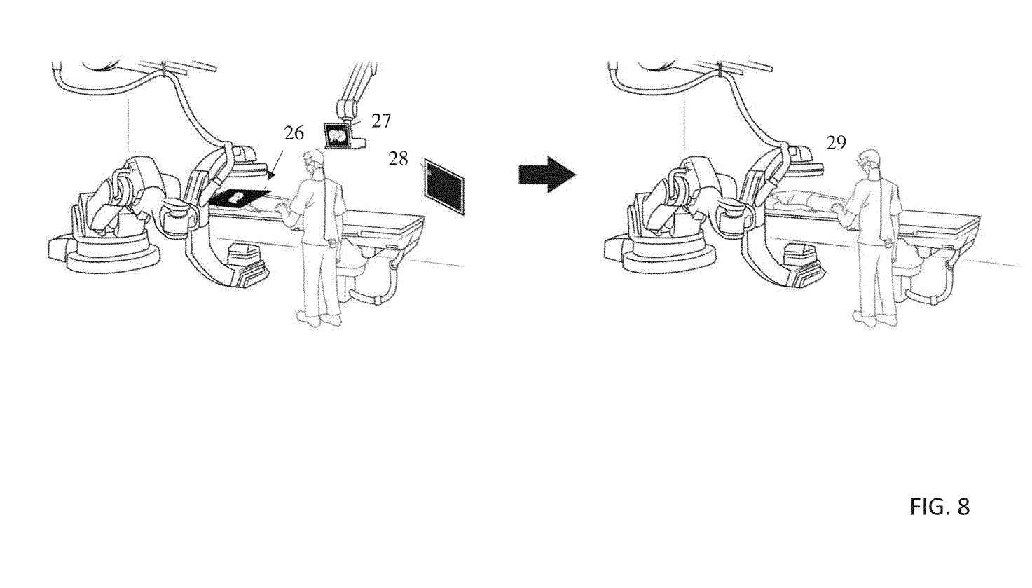

FIG. 8 is an exemplary figure illustrating that at any time the operator is able to remove the screens 26, 27, 28 from view which may mean minimizing the screens, hiding all of the information, or moving them to the side but the processes will continue to run in the background. At any user desired time point the screens can reappear when queued by the user using voice control, a virtual button, or any other signaling mechanism including set on a timer 29.

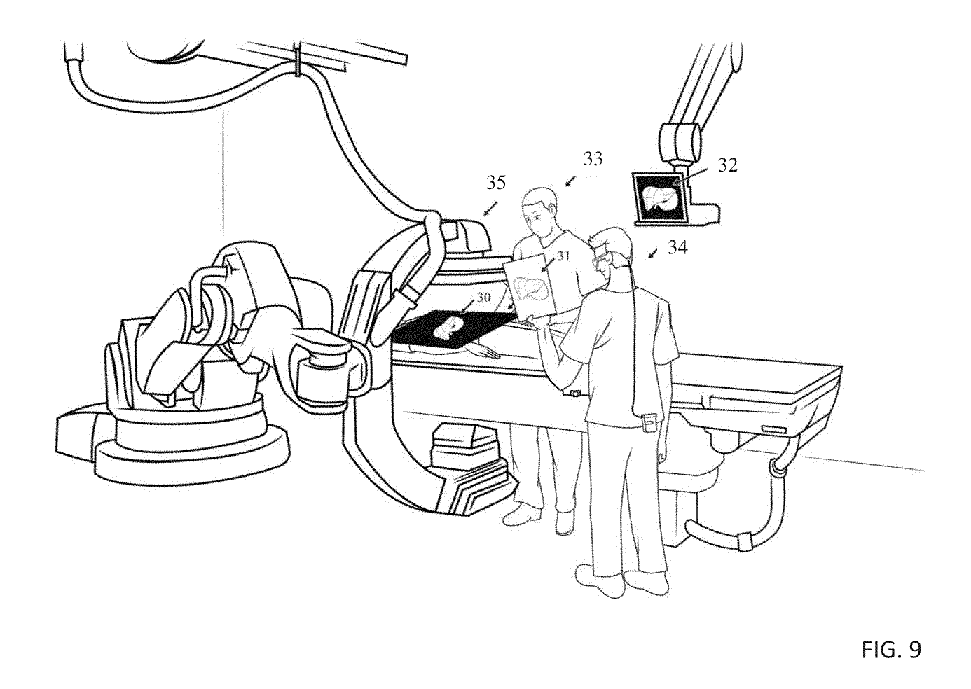

FIG. 9 is an exemplary figure that illustrates exemplary collaboration between operators. An operator 34 can select or deselect an image and annotate the image using their hand or finger location and save a drawing 31 in 2-dimensional or 3-dimensional space. This data can be stored locally or distributed through the central processing hub. Additionally, the device will have the capabilities to transmit information, be it auditory, visual, or information derived from any other sensor on the device, to be viewable on another device 30, 31, 32. This can be achieved either through communication through a central hub or direct transfer of information from one device to another using the protocols such as WebRTC described above.

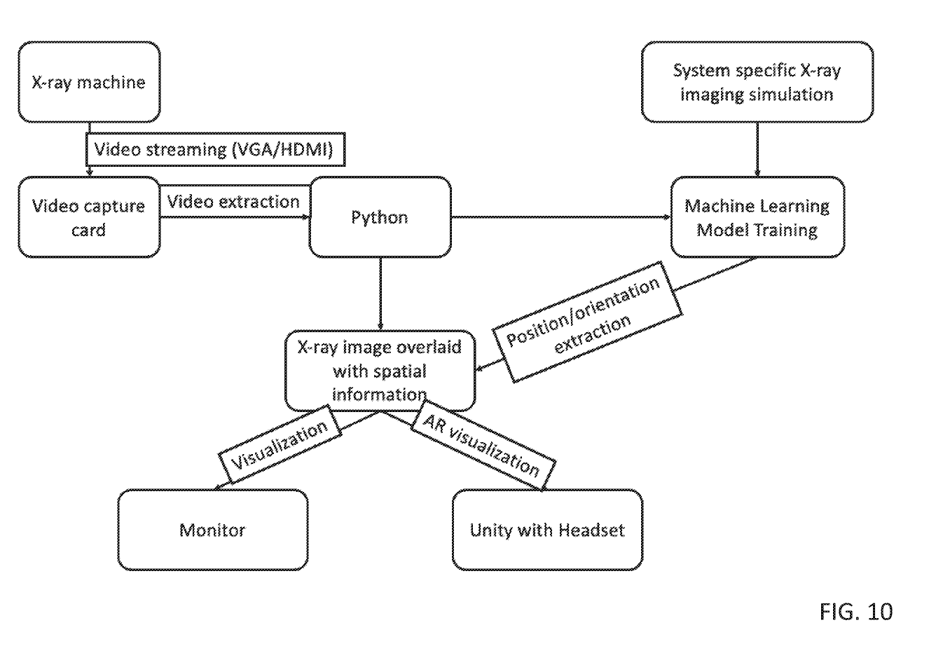

FIG. 10 is an exemplary embodiment of an image processing system and methods including one or more machine learning (ML) models and/or techniques. Two methods are included in this exemplary embodiment. One method is for an independent mobile application (e.g., executable by a mobile computing device such as a smartphone, tablet computing device, etc.). ML models trained in a ML model training environment 37 (e.g., TensorFlow, Caffe, scikit-learn, or any other machine learning software) are integrated with the ML model 38 (e.g., CoreML model, etc.), which is usable in augmented reality (AR) platform 39 (e.g., ARKit, etc.). In one embodiment the bear platform 39 is installed and run (e.g., executed) independently on the mobile device. In another embodiment the ML models are server based 41. In this embodiment, ML tools are installed and run (e.g., executed) on one or more servers with one or more high-performance CPUs and/or GPUs. Live streaming is used to transfer images and other information between server and mobile devices using protocols including but not limited to the protocols described above.

In an embodiment, the position-orientation determination program is implemented in Python. Video capture card is used to stream the X-ray video from the imaging device to the computer. The streamed X-ray frames are read into Python and analyzed by the ML model through Python interface of TensorFlow. The results are merged with the original frame for visualization. TensorFlow is not necessary for this specific model. Nevertheless, it makes it trivial to import other readily available ML models for real time analysis.

An example of a ML algorithm based on the framework in FIG. 10, for bone segmentation, is described. Bony landmarks such as the spinal and hip bones may be annotated manually on a single slice. Subsequently, the image would be registered to a phantom based on bony features. Registration means recognizing certain imaging features and correlating them to features on the patient's body so that the images would be a rough approximation of the patient's anatomy. The algorithm then identifies the remainder of the bone shape and registers to the patient's anatomy. Independent bony structures (e.g., the spine) may have similar shapes and be hard to differentiate, but when viewed in relation to surrounding structures, there are fewer features to match. Starting with sagittal views of the spines from phantom and the patient, the images will be registered first at sacral levels based on its unique shape, then segment by segment superiorly. Two images will thus be registered in sagittal view, based on which complete 3D registration can be used to improve the accuracy. ML algorithms could include processes as described above but also other areas of medical application such as medical registration, diagnosis, angular adjustment, or segmentation.

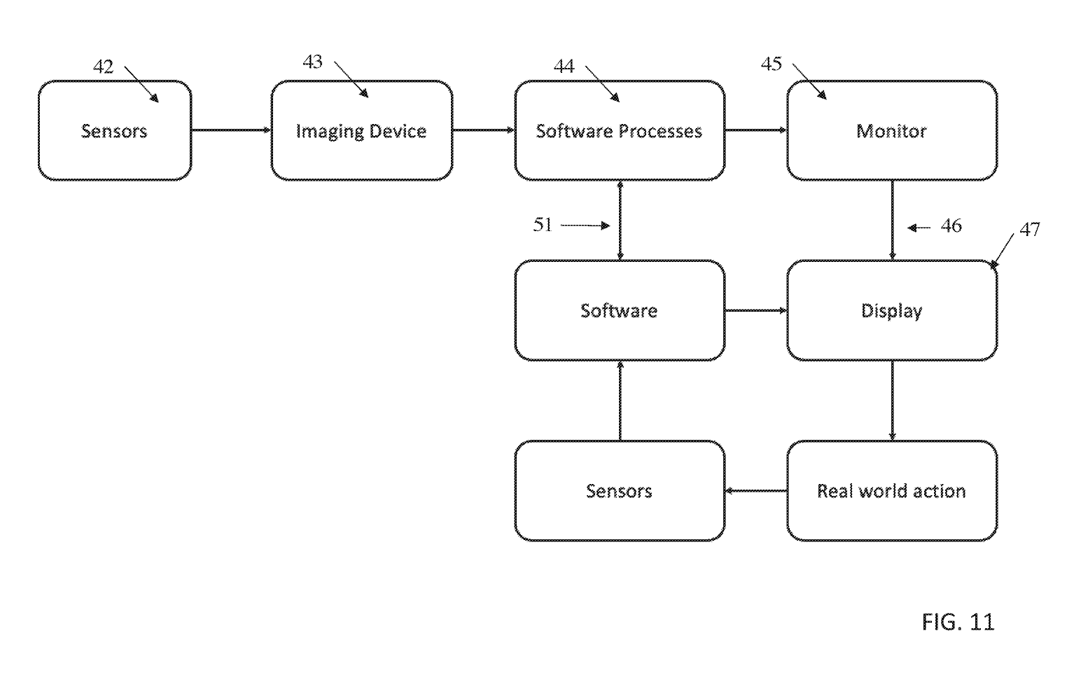

FIG. 11 is an exemplary illustration of an application-programming interface (API) between the software of an imaging device and that of an augmented reality device. The data flow would follow the standard framework with sensors 42 providing data input into the radiology imaging device 43 which would interpret the data using its software processes 44 and display the information on a monitor 45. The API standardizes the flow of information 46 from the display 45 to the headset 47 and then from the headset software back to the imaging device software 51. Each vendor encodes information within their own proprietary data structure which would make this API would standardize interoperability. Third-party application developers would be able to build an AR app for any headset and that would work for any imaging device.

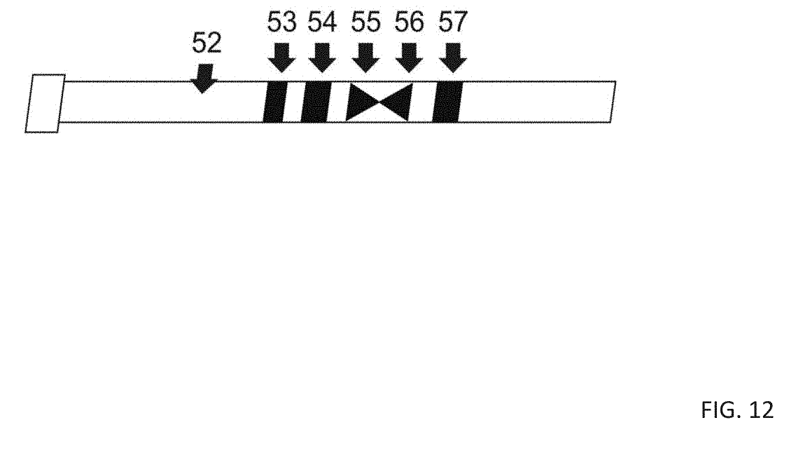

FIG. 12 is an illustration of a catheter or needle or any other long tubular device 52 with radiopaque markers arranged in a pattern. This specific pattern consisting of sharp vertices 53 and 54, and asymmetric pattern 55, 56, 57 can be used in conjunction with image processing algorithms to determine the position of the catheter and in turn guide interventions. The pattern can occur anywhere along the length of the device, but in an embodiment is placed near the terminating tip. An example of using the image processing algorithm would be the determination of the orientation of the radiopaque markers and in turn the position of the catheter to guide interventions.

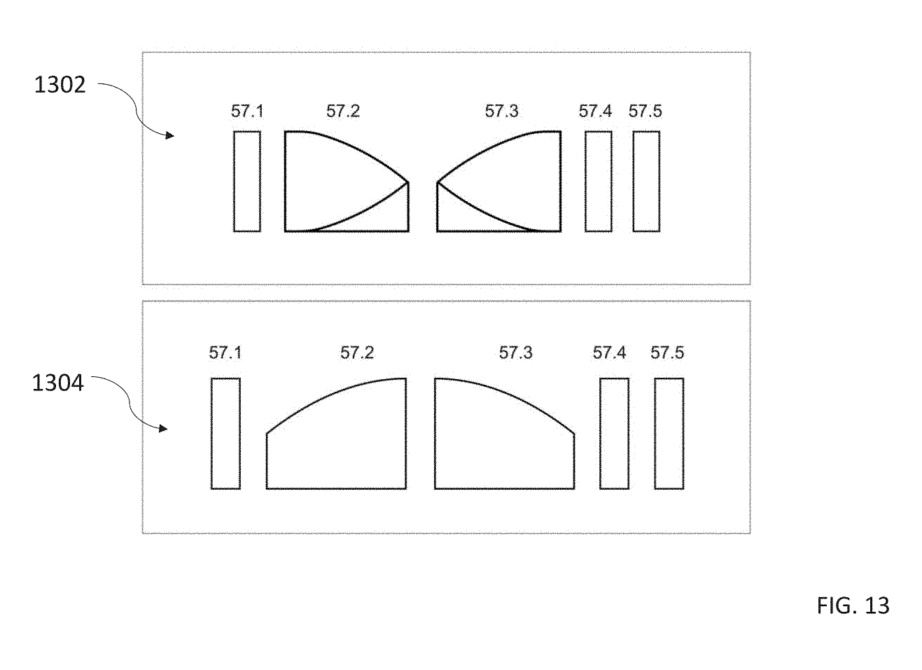

FIG. 13 shows various orientations of a three-dimensional alternative embodiment using a curvilinear arrangement. The orientation at 1302 is an example around the long axis. The orientation at 1304 shows the same example of 1302 with its central axis horizontal and then another view around that axis to show the differing two-dimensional projected result.

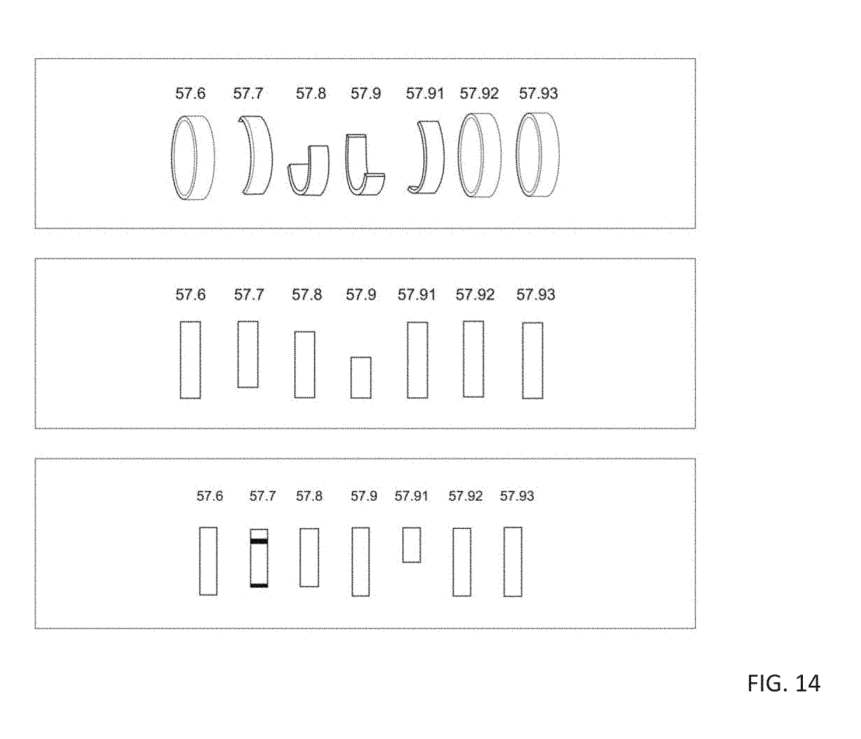

FIG. 14 shows a different configuration of discrete radiopaque rings in different orientations. The heights of the rings 57.7, 57.8, 57.9, 57.91 can be used in image processing and machine learning algorithms described herein for various purposes including, but not limited to, the description of position of the surgical device and its relation to other anatomical and pathological features. The number of rings can vary from one to more than 15 depending on application. On constructions utilizing more than one ring, at least one ring, usually placed on the end of the arrangement but not limited to that location, will serve as a reference for the other rings in calculation. This arrangement can be implemented without that reference ring as well.

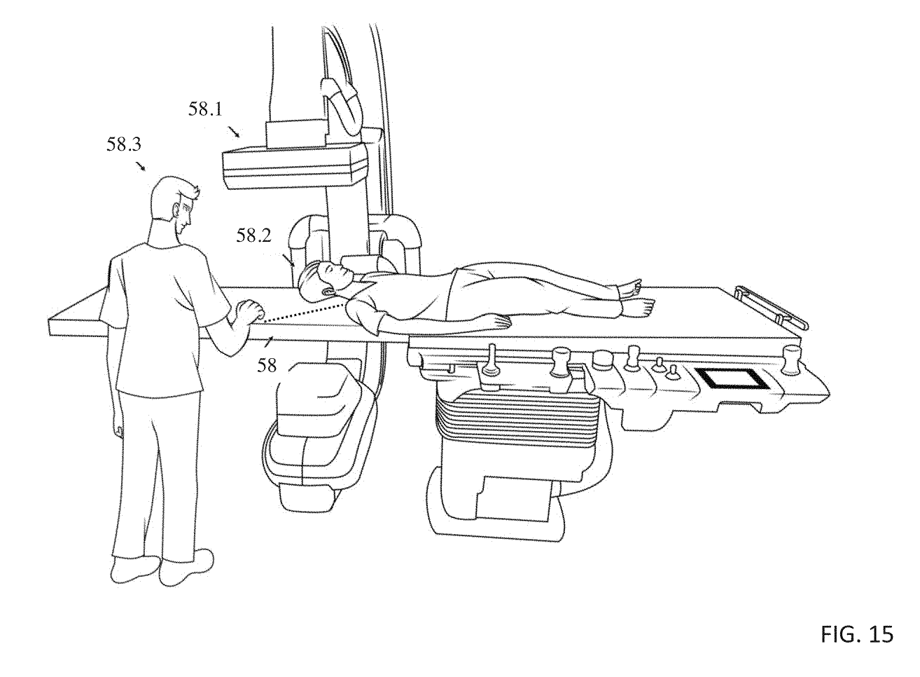

FIG. 15 illustrates a catheter, needle, or other device with radiopaque markers being used in an interventional procedure. The device may be used in the same manner as current existing surgical tools, but the image processing algorithms described herein would run at the user's direction, or automatically at preset points, to deliver operational guidance. The catheter 58 is inserted into the patient and fluoroscopic images are taken using the image intensifier 58.1. The radiopaque markers will show up on a 2D image. The machine learning algorithm can use this pattern for analysis, additionally the 2D pattern can also be used as a target marker to overlay augmented reality objects.

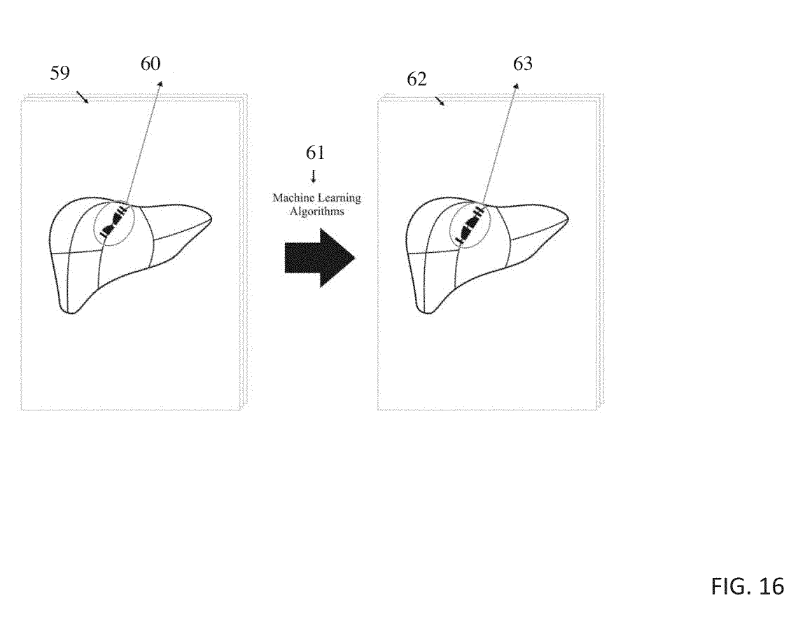

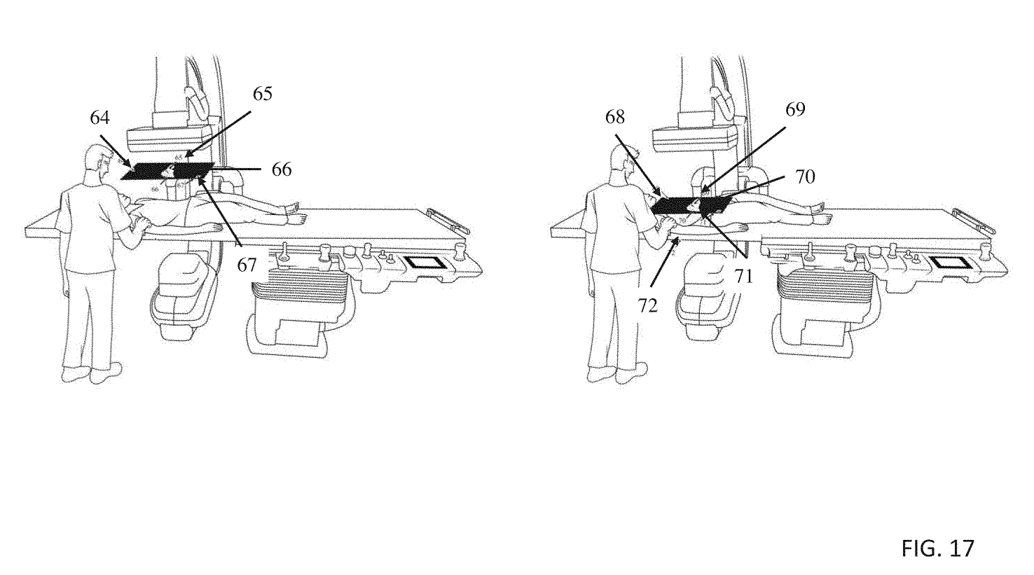

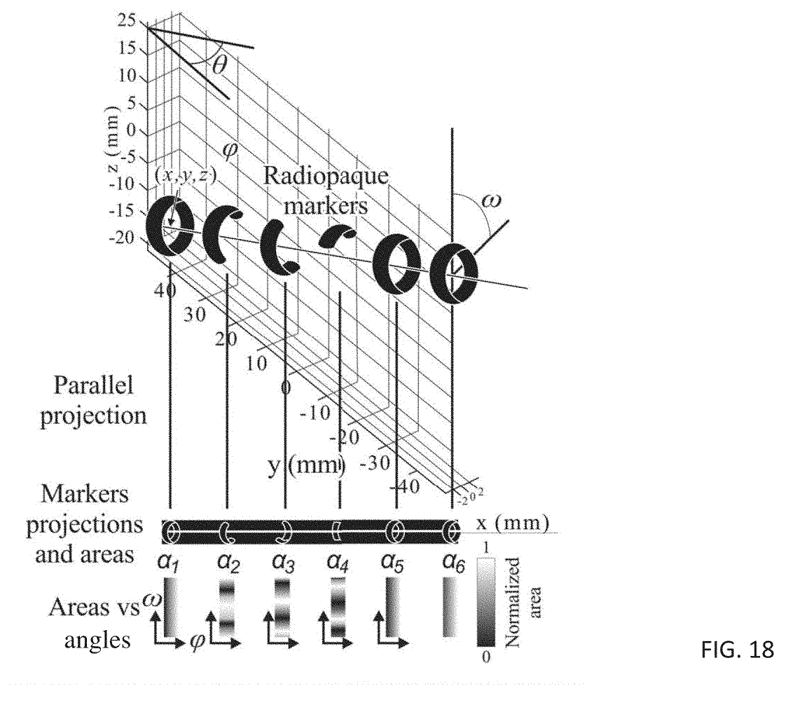

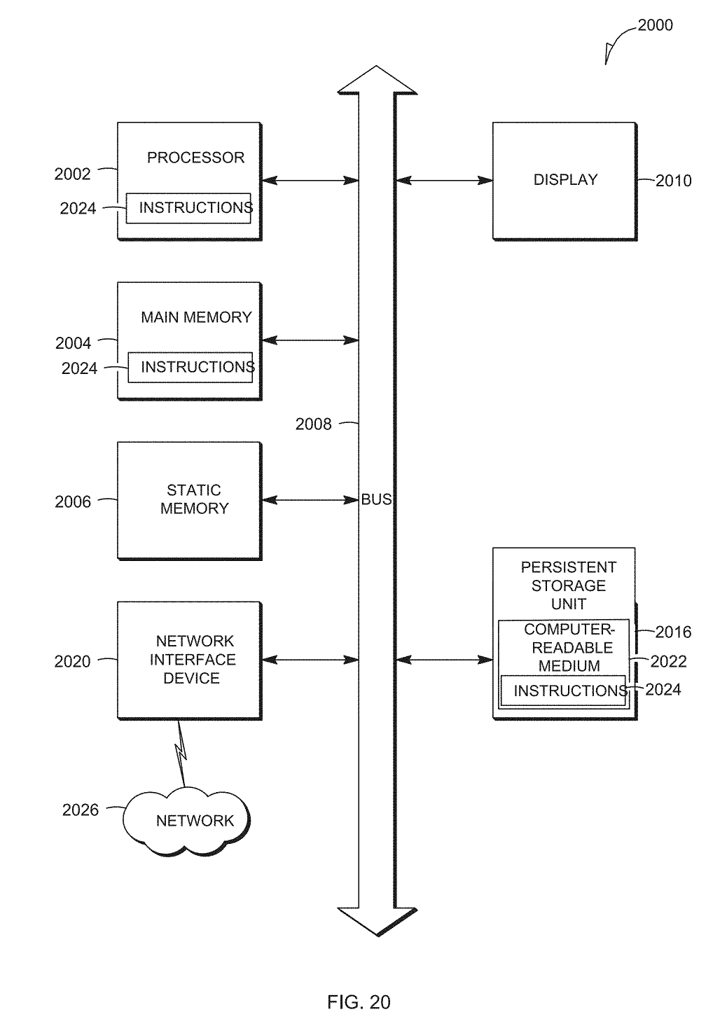

FIG. 16 is an illustration of the image-processing algorithm detecting the vertices of the radiopaque markers. This will be further explained in FIGS. 18-20. FIG. 17 is an illustration of the system in use with a radiopaque target marker 71 and catheter/needle with radiopaque markers impregnated into its structure. The system will be able to overlay the images over the radiopaque target marker in near anatomic position to aid the intervention.

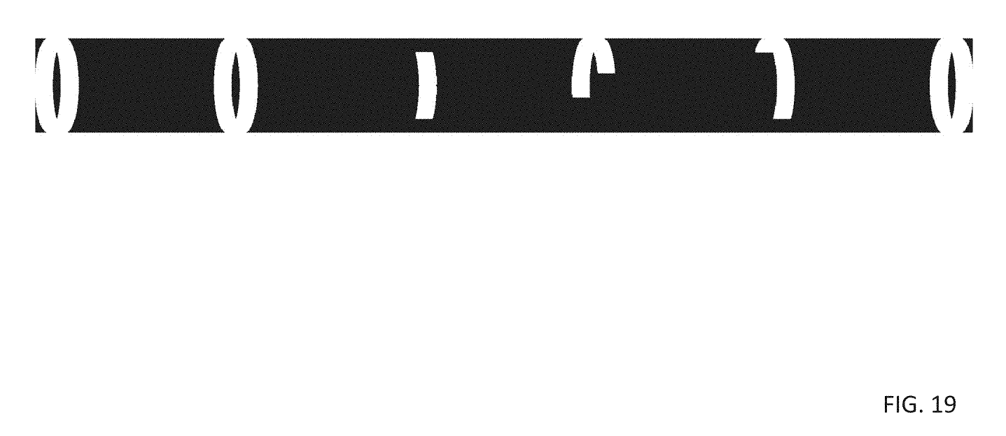

In an example of a method of determining the orientation from such a marker, FIG. 18 shows a six-part example of such a marker, with three reference subsections on the end and three shaped subsections between, placed in three dimensional space. The structure in FIG. 18 is projected downwards to the x-y plane to simulate the result from the procurement of an image of such device, creating a data set similar to FIG. 19. The direction of compression is not specific, but serves as a reference for the desired data and can be arbitrarily chosen. The markers are identified due to the sharp contrast beyond a chosen value in the resulting projection, and the areas of the subsections are recorded. The six sub-areas are compared to known area values for each sub-section calculated from the original design, shown in FIG. 18. The six measured areas will only match in each subsection for the same value of the angles (φ, ω) at one location, if designed properly. The two calculated matching angles can be recorded for further processing.

Parallel projection is used as an example, as shown in FIG. 18. Six radiopaque markers (illustrated) are attached to a catheter (not illustrated). The systems and methods described herein rotate the catheter around its axis by angle co and rotate the catheter axis with angle φ from the projection axis. The markers' projections in the X-ray image are simulated and their areas (a₁, a₂, a₃, a₄, a₅, a₆) are calculated. Based on the simulation, we find different (φ, ω) leads to different (a₁, a₂, a₃, a₄, a₅, a₆). Storing the simulated mapping, we can recover (φ, ω) simply by referring to (a₁, a₂, a₃, a₄, a₅, a₆). The catheter axis' projection in the X-ray image can be easily identified though the centers of the three rings with complete ring shape (one at one end and two at the other end). Then angle θ and the projection location (x, y) can be calculated. Exemplary aspects of image processing including projection are described in Appendix A.

Parallel projection is simple but loses the depth information. Unparallel projections can also be used to encode depth (z) information. Then (φ, ω) should be replaced by (z, φ, ω). More markers can be used and the same simulation could be done to build the mapping from spatial information to areas. The mapping will be used to recover spatial information in practice.

This process can be aided by taking other projections or two-dimensional images from known angles and correlating the data. Other ways to calculate the orientation of the device include but are not limited to means of measuring section widths, heights, volumes, areas, identifying special features on the markers, or through comparing multiple projection combinations of such listed means.

With reference to FIG. 10, an example embodiment extends to a machine in the example form of a computer system 2000 within which instructions for causing the machine to perform any one or more of the methodologies discussed herein may be executed. For example, computer system 2000 may comprise, in whole or in part, aspects of system 100 (e.g., processor(s) 106, data recording and storage device(s) 108, visual display device(s) 110, etc.). In alternative example embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. Further, while only a single machine is illustrated, the term “machine” shall also be taken to include any collection of machines that individually or jointly execute a set (or multiple sets) of instructions to perform any one or more of the methodologies discussed herein.

The example computer system 2000 may include at least one processor 2002 (e.g., a central processing unit (CPU), a graphics processing unit (GPU), or both), a main memory 2004 and a static memory 2006, which communicate with each other via a bus 2008. In some embodiments, processor 2002 may comprise, in whole or in part, processor(s) 106. In some embodiments, main memory 2004 may comprise, in whole or in part, data recording and storage device(s) 108. The computer system 2000 may further include a touchscreen display unit 2010. In example embodiments, the computer system 2000 also includes a network interface device 2020.

The persistent storage unit 2016 includes a machine-readable medium 2022 on which is stored one or more sets of instructions 2024 and data structures (e.g., software instructions) embodying or used by any one or more of the methodologies or functions described herein. The instructions 2024 may also reside, completely or at least partially, within the main memory 2004 or within the processor 2002 during execution thereof by the computer system 2000, the main memory 2004 and the processor 2002 also constituting machine-readable media. In some embodiments, instructions 2024 comprise, in whole or in part, the ML algorithm of FIG. 10, the API of FIG. 11, and/or image processing and machine learning algorithms further described herein. Additionally or alternatively, the persistent storage unit 2016 may comprise, in whole or in part, data recording and storage device(s) 108 in some embodiments.

While the machine-readable medium 2022 is shown in an example embodiment to be a single medium, the term “machine-readable medium” may include a single medium or multiple media (e.g., a centralized or distributed database, or associated caches and servers) that store the one or more instructions. The term “machine-readable medium” shall also be taken to include any tangible medium that is capable of storing, encoding, or carrying instructions for execution by the machine and that cause the machine to perform any one or more of the methodologies of embodiments of the present invention, or that is capable of storing, encoding, or carrying data structures used by or associated with such instructions. The term “machine-readable storage medium” shall accordingly be taken to include, but not be limited to, solid-state memories and optical and magnetic media that can store information in a non-transitory manner, i.e., media that is able to store information. Specific examples of machine-readable storage media include non-volatile memory, including by way of example semiconductor memory devices (e.g., Erasable Programmable Read-Only Memory (EPROM), Electrically Erasable Programmable Read-Only Memory (EEPROM), and flash memory devices); magnetic disks such as internal hard disks and removable disks; magneto-optical disks; and CD-ROM and DVD-ROM disks. A machine-readable storage medium does not include signals.

The instructions 2024 may further be transmitted or received over a communications network 2026 using a signal transmission medium via the network interface device 2020 and utilizing any one of a number of well-known transfer protocols (e.g., FTP, HTTP). Examples of communication networks include a local area network (LAN), a wide area network (WAN), personal area network (PAN), wireless personal area network (WPAN), the Internet, mobile telephone networks, Plain Old Telephone (POTS) networks, and wireless data networks (e.g., WiFi and WiMax networks). The term “machine-readable signal medium” shall be taken to include any transitory intangible medium that is capable of storing, encoding, or carrying instructions for execution by the machine, and includes digital or analog communications signals or other intangible medium to facilitate communication of such software. In some embodiments, communications network 2026 comprises, in whole or in part, the electrical and/or communicative couplings described herein.

From the foregoing, it will be seen that this invention is one well adapted to attain all the ends and objects hereinabove set forth together with other advantages which are obvious and which are inherent to the structure. It will be understood that certain features and sub combinations are of utility and may be employed without reference to other features and sub combinations. This is contemplated by and is within the scope of the claims. Since many possible embodiments of the invention may be made without departing from the scope thereof, it is also to be understood that all matters herein set forth or shown in the accompanying drawings are to be interpreted as illustrative and not limiting.

The constructions described above and illustrated in the drawings are presented by way of example only and are not intended to limit the concepts and principles of the present invention. Thus, there has been shown and described several embodiments of a novel invention. As is evident from the foregoing description, certain aspects of the present invention are not limited by the particular details of the examples illustrated herein, and it is therefore contemplated that other modifications and applications, or equivalents thereof, will occur to those skilled in the art. The terms “having” and “including” and similar terms as used in the foregoing specification are used in the sense of “optional” or “may include” and not as “required”. Many changes, modifications, variations and other uses and applications of the present construction will, however, become apparent to those skilled in the art after considering the specification and the accompanying drawings. All such changes, modifications, variations and other uses and applications which do not depart from the spirit and scope of the invention are deemed to be covered by the invention which is limited only by the claims which follow.

What is claimed is:

1. A system, comprising:
a visual input sensor, wherein the visual input sensor is operable to capture a live stream video of a patient;
an image interface, wherein the image interface is operable to receive image data of the patient from a plurality of different imaging devices;
at least one visual display device;
at least one processor; and
at least one non-transitory computer-readable storage medium, wherein the at least one non-transitory computer-readable storage medium stores one or more processor-executable instructions that, when executed by the at least one processor:
receive the live stream video of the patient,
receive the image data of the patient,
determine an orientation of the patient from the live video stream,
generate a video output of the live video stream for outputting via the at least one visual display device, and
overlay the image data of the patient in the video output, wherein the overlaid image data is aligned with the orientation of the patient.

2. The system of claim 1, further comprising a touchless user input sensor, wherein the touchless user input sensor is operable to receive a touchless input from an operator to manipulate at least one of the video output and the overlaid image data.

3. The system of claim 2, wherein the touchless input includes at least one of eye tracking, gesture tracking, and speech.

4. The system of claim 1, further comprising a network interface to communicate the video output and the overlaid image data to one or more other visual display devices, thereby enabling collaboration among a plurality of operators.

5. The system of claim 1, wherein the image data includes data representing one or more radiopaque markers arranged on a surgical tool, and wherein the at least one non-transitory computer-readable storage medium further stores one or more processor-executable instructions that, when executed by the at least one processor:
determine an orientation of the radiopaque markers;
determine a position of the surgical tool relative to the patient from the orientation of the radiopaque markers; and
overlay a visual representation of the surgical tool at the determined position of the patient in the video output.

6. A method, comprising:
capturing a live stream video of a patient via at least one visual input sensor;
receiving image data of the patient from a plurality of different imaging devices;
determining an orientation of the patient from the live video stream;
generating a video output of the live video stream;
overlaying the image data of the patient in the video output, wherein the overlaid image data is aligned with the orientation of the patient; and
outputting, via at least one visual display device, the video output and overlaid image data.

7. The method of claim 6, further comprising receiving, via a touchless user input sensor, a touchless user input from an operator to manipulate at least one of the video output and the overlaid image data.

8. The method of claim 7, wherein the touchless input includes at least one of eye tracking, gesture tracking, and speech.

9. The method of claim 6, further comprising communicating, via network interface, the video output and the overlaid image data to one or more other visual display devices, thereby enabling collaboration among a plurality of operators.

10. The method of claim 6, further comprising:
receiving image data representing one or more radiopaque markers arranged on a surgical tool;
determining an orientation of the radiopaque markers;
determining a position of the surgical tool relative to the patient from the orientation of the radiopaque markers; and
overlaying a visual representation of the surgical tool at the determined position of the patient in the video output.

11. A non-transitory computer readable storage medium comprising a set of instructions executable by a computer, the non-transitory computer readable storage medium comprising:
instructions for capturing a live stream video of a patient via at least one visual input sensor;
instructions for receiving image data of the patient from a plurality of different imaging devices;
instructions for determining an orientation of the patient from the live video stream;
instructions for generating a video output of the live video stream;
instructions for overlaying the image data of the patient in the video output, wherein the overlaid image data is aligned with the orientation of the patient; and
instructions for outputting, via at least one visual display device, the video output and overlaid image data.

12. The non-transitory computer readable storage medium of claim 11, further comprising instructions for receiving, via a touchless user input sensor, a touchless user input from an operator to manipulate at least one of the video output and the overlaid image data.

13. The non-transitory computer readable storage medium of claim 12, wherein the touchless input includes at least one of eye tracking, gesture tracking, and speech.

14. The non-transitory computer readable storage medium of claim 11, further comprising instructions for communicating, via network interface, the video output and the overlaid image data to one or more other visual display devices, thereby enabling collaboration among a plurality of operators.

15. The non-transitory computer readable storage medium of claim 11, further comprising:
instructions for receiving image data representing one or more radiopaque markers arranged on a surgical tool;
instructions for determining an orientation of the radiopaque markers;
instructions for determining a position of the surgical tool relative to the patient from the orientation of the radiopaque markers; and
instructions for overlaying a visual representation of the surgical tool at the determined position of the patient in the video output.
